(19) United States
(12) Patent Application Publication
(10) Pub. No.: US 2004/0110770 A1
(43) Pub. Date: Jun. 10, 2004

(54) Oxindoles which are inhibitors of CDK-1 and their application in therapeutics

(75) Inventors: Jean-Francois Riou, Reims (FR); Michel Maratrat, Villeneuve Le Roi (FR); Lucile Grondard, Courcouronnes (FR); Fabienne Thompson, Paris (FR); Odile Petitgenet, Paris (FR); Patrick Mailliet, Fontenay Sous Bois (FR); Jacques Lavayre, Marolles En Brie (FR)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: AVENTIS PHARMA SA, Antony (FR)

(21) Appl. No.: 10/644,411

(22) Filed: Aug. 20, 2003

Related U.S. Application Data
(63) Continuation-in-part of application No. PCT/FR02/00681, filed on Feb. 25, 2002.

(30) Foreign Application Priority Data
Feb. 27, 2001 (FR) 01 02624

(51) Int. Cl. A61K 31/506; A61K 31/4439; A61K 31/4178; A61K 31/404; C 07D 4 3/02

(52) U.S. Cl. 514/256; 514/397; 514/339; 514/414; 544/333; 546/277.7; 548/312.1; 548/465

(57) ABSTRACT

The present invention relates to a compound of formula (I):

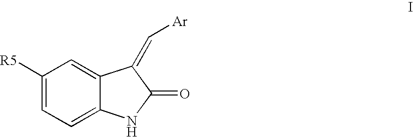

wherein

R5 is selected from the group consisting of 3-pyridyl, 5-pyrimidinyl, —CONH—(C₁-C₄ alkyl), —NHCO—(C₁-C₄ alkyl), halogen, —SO₂NH₂, —NO₂, —CF₃ or thien-2-ylcarbonyl

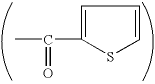

and —CO₂R where R can be hydrogen or C₁-C₄ alkyl; and

Ar is selected from the group consisting of 5-imidazolyl, 2-pyrrolyl optionally substituted by a C₁-C₄ alkyl radical, 2-furyl or 2-thiazolyl,

in the E or Z geometrical isomeric form or a mixture of the two geometrical isomeric forms.

The invention is also directed to a method of treating primary and secondary tumours in a patient in need thereof comprising administering to the patient a therapeutically effective amount of a compound of formula I. The invention is also directed to a method of using a compound of formula I to treat cancer, inhibit the proliferation of a cell and induce cell apoptosis, comprising contacting a cell with an effective amount of the compound of formula I., The invention is also directed to a method of preparing the compound of formula I.

### CROSS REFERENCE

This application is a continuation-in part of International Application No. PCT/FR02/00681, filed Feb. 25, 2002.

### BACKGROUND OF THE INVENTION

The present invention relates to a family of oxindoles which are chemical inhibitors of the Cdc2/cyclin B (CDK-1) enzymatic complex and to their application in therapeutics.

### FIELD OF THE INVENTION

As is well known, the cell cycle of a eukaryote comprises different stages (see FIG. 1):

After the M phase, which is composed of a nuclear division (mitosis) and of a cytoplasmic division (cytodieresis), the daughter cells begin the interphase of a new cycle. This interphase begins with the G1 phase, during which an increased resumption of the biosynthetic activities of the cell is recorded. The S phase begins when the synthesis of DNA begins and terminates when the chromosomes have replicated (each chromosome is then composed of two identical sister chromatids). The cell subsequently enters the G2 phase (final phase of the interphase), which continues until mitosis begins, initiating the M phase. During this phase, the sister chromatids separate, two new nuclei are formed and the cytoplasm divides to give two daughter cells each possessing a nucleus. Cytodieresis terminates the M phase and marks the beginning of the interphase of the following cell cycle.

The molecular machinery of the cell cycle is composed of regulatory factors which control the progression in the cell cycle. The passage from one phase to another in a cycle is under the control of a family of small protein serine/threonine kinases, the cyclin-dependent kinases (CDKs), which regulate the activity of proteins by phosphorylation. The rate of expression of the CDKs is more or less constant during the cell cycle, but these protein kinases are inactive in themselves and have to be activated in order to acquire kinase activity. The enzymatic activity and the specificity of the CDKs depend on their association with a regulatory subunit belonging to the family of cyclins.

The entry into mitosis, in particular, is under the control of an M-phase promoting factor (or MPF) which is composed of the combination of a molecule of (CDK1=p34cdc2) and of a molecule of cyclin B. The activated cyclin B-CDK1 complex makes possible the G2/M transition by phosphorylating numerous substrates.

Thus, the modulation of the activity of the cyclin B-CDK1 complex is a key mechanism in halting cell proliferation and CDKs constitute favoured molecular targets in the search for selective inhibitors of cell proliferation. This is because some properties of CDKs (in particular the very frequent detrimental changes, in human tumours, of CDKs and of their regulators) and of their natural protein inhibitors have encouraged the search for chemical inhibitors of CDK for the purpose of antitumour applications.

Numerous compounds have thus been tested and recognized as CDK inhibitors: purines, paullones, flavopiridol, indirubins, olomoucine, roscovitine, 1-butyrolactone, toyocamycin and others. The chemical inhibitors which act more particularly on the cyclin B/CDK1 complex in fact prevent the phosphorylation of substrates such as histone H1, the γ and δ subunits of the elongation factor or vimentin.

Thus, the first chemical inhibitors of CDK exhibit advantageous properties that justify their evaluation as potential anticancer products and the continuation of the search for new, more effective, molecules.

Patent Application WO 96/40116 discloses oxindoles used for the purpose of modulating the transduction signal of protein tyrosine kinase (PTK).

### SUMMARY OF THE INVENTION

The present invention is directed to a family of compounds of the family of the oxindoles having a marked action on cell proliferation and on CDK-1. That family is directed to novel oxindole compounds corresponding to the formula (I):

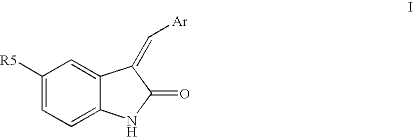

wherein

R5 is selected from the group consisting of 3-pyridyl, 5-pyrimidinyl, —CONH—(C₁-C₄ alkyl), —NHCO—(C₁-C₄ alkyl), halogen, —SO₂NH₂, —NO₂, —CF₃ or thien-2-ylcarbonyl

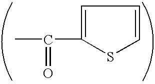

and —CO₂R where R can be hydrogen or C₁-C₄ alkyl; and

Ar is selected from the group consisting of 5-imidazolyl, 2-pyrrolyl optionally substituted by a C₁-C₄ alkyl radical, 2-furyl or 2-thiazolyl,

in the E or Z geometrical isomeric form or a mixture of the two geometrical isomeric forms.

The invention is also directed to the method of using the compounds as a medicament, and method for their preparation.

### BRIEF DESCRIPTION OF THE DRAWING

FIG. 1 shows different stages of the cell cycle of a eukaryote.

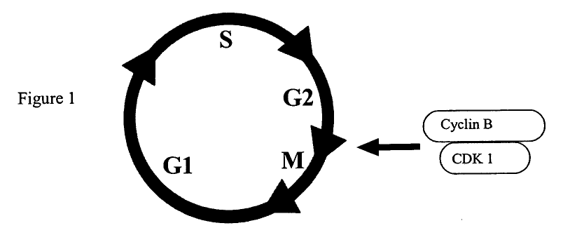

### DETAILED DESCRIPTION OF THE INVENTION

Embodiments of the Invention

A preferred embodiment of the invention is the compounds of formula (I), wherein R5 is 3-pyridyl, —CONH-methyl or —NHCO-methyl.

Another preferred embodiment of the invention is the compounds of formula (I), wherein Ar is 5-imidazolyl or 5-(4-methylimidazolyl).

Another embodiment according to the invention is the compound of formula (I) selected from group of formulae consisting of:

1,3-dihydro-3-(imidazol-4-ylmethylene)-5-(pyrid-3-yl)-2H-indolin-2-one;

1,3-dihydro-3-(pyrrol-2-ylmethylene)-5-(pyrid-3-yl)-2H-indolin-2-one;

1,3-dihydro-3-(imidazol-4-ylmethylene)-5-(N-methylcarboxamido)-2H-indolin-2-one; and

1,3-dihydro-3-(imidazol-4-ylmethylene)-5-(acetylamino)-2H-indolin-2-one.

The preparation of the compound of formula (I) in which R5 and Ar have the abovementioned meanings according to the invention is by coupling an indolin-2-one of formula (II), wherein R5 has the abovementioned meaning, with an aromatic aldehyde of general formula (III) wherein Ar has the abovementioned meaning, according to the scheme below:

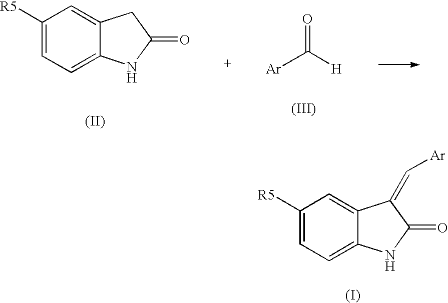

The coupling reaction is generally carried out under the conditions described by E. Knoevenagel (Chem. Ber. 1900, 23, 1972), namely in a protic solvent, such as methanol or ethanol, in the presence of a catalytic amount of organic base, such as piperidine, at a temperature of between 20° C. and the reflux temperature of the solvent used.

The indolin-2-ones of formula (II) and the aromatic aldehydes of formula (III) wherein R5 and Ar respectively have the abovementioned meanings are either commercially available or are prepared according to the conditions described in the literature.

Other aspects of the invention will become apparent in the light of the description herein and examples that follow.

The Applicant Company has discovered that the compounds of formula (I) in accordance with the invention have properties of inhibiting protein kinases (CDKs). These proteins play a key role in the initiation, development and completion of the events of the cell cycle. Thus, molecules that inhibit CDK are capable of limiting inopportune cell proliferations, such as those observed in cancers.

The protein kinase CDK1 is particularly sensitive to the inhibitory effects of the compounds of the present invention.

In addition to their inhibitory properties specific to the protein kinase CDK-1, the products of the present invention additionally have cell effects, such as antiproliferative properties, by blocking cell division during the cycle, and apoptotic properties, by induction of cell apoptosis.

The compound in accordance with the invention is useful as an anticancer therapeutic, i.e., in the treatment of primary and secondary tumours.

A compound of the invention can also be used alone or in combination with treatments such as chemotherapy, radiotherapy or antiangiogenic treatments optionally employing other active substances. The invention applies to pharmaceutical compositions comprising, as active principle, at least one compound of the formula (I) as defined above in a pharmaceutically acceptable medium.

Pharmaceutical compositions can be administered by the buccal route, parenteral route or local route, as a topical application to the skin or mucous membranes, or by injection by the intravenous or intramuscular route. These compositions can be solid or liquid and can be provided in any of the pharmaceutical forms commonly used in human medicine, such as, for example, simple or sugar-coated tablets, pills, lozenges, gelatin capsules, drops, granules, injectable preparations, ointments, creams or gels. They are prepared according to the usual methods. The active principle can be incorporated therein with excipients commonly employed in these pharmaceutical compositions, such as talc, gum arabic, lactose, starch, magnesium stearate, cocoa butter, aqueous or nonaqueous vehicles, fatty substances of animal or vegetable origin, paraffin derivatives, glycols, various wetting, dispersing or emulsifying agents, or preservatives.

The usual dosage, which can vary according to the product used and the subject treated, can be, for example, from 0.05 to 5 grams per day for adults.

### EXPERIMENTAL

Preparation examples are described below and are illustrative of the invention without, however, limiting it:

### Example 1

### 1,3-dihydro-3-(imidazol-4-ylmethylene)-5-(pyrid-3-yl)-2H-indolin-2-one

0.28 g (3 mmol) of imidazole-4-carboxaldehyde is added to a solution of 0.61 g (3 mmol) of 5-(pyrid-3-yl)-2H-indolin-2-one in 75 ml of ethanol comprising 0.02 ml of piperidine. The reaction medium is brought to reflux for 4 hours. After cooling, the precipitate formed is filtered off, washed with 2 times 5 ml of ice-cold ethanol and dried under reduced pressure. 0.62 g (75%) of 1,3-dihydro-3-(imidazol-4-ylmethylene)-5-(pyrid-3-yl)-2H-indolin-2-one is thus obtained in the form of a lemon yellow solid, the characteristic of which is as follows: melting point=310° C.

### Example 2

### 1,3-dihydro-3-(pyrrol-2-ylmethylene)-5-(pyrid-3-yl)-2H-indolin-2-one

By carrying out the preparation as in Example 1 but using 2.1 g (10 mmol) of 5-(pyrid-3-yl)-2H-indolin-2-one in 150 ml of ethanol and from 0.99 g (10 mmol) of pyrrole-2-carboxaldehyde, 2.66 g (92.5%) of 1,3-dihydro-3-(pyrrol-2-ylmethylene)-5-(pyrid-3-yl)-2H-indolin-2-one is obtained in the form of an orange solid, the characteristic of which is as follows: melting point=225° C.

### Example 3

### 1,3-dihydro-3-(imidazol-4-ylmethylene)-5-(N-methylcarboxamido)-2H-indolin-2-one

By carrying out the preparation as in Example 1 but from 0.2 g (1.05 mmol) of 5-(methylcarboxamido)-2H-indolin-2-one in 25 ml of ethanol and from 0.1 g (1.04 mmol) of imidazole-5-carboxaldehyde, 0.21 g (84%) of 1,3-dihydro-3-(imidazol-4-ylmethylene)-5-(N-methylcarboxamido)-2H-indolin-2-one is obtained in the form of an orange solid, the characteristic of which is as follows: melting point >260° C.

### Example 4

### 1,3-dihydro-3-(imidazol-4-ylmethylene)-5-(acetylamino)-2H-indolin-2-one

By carrying out the preparation as in Example 1 but from 0.4 g (2.1 mmol) of 5-(acetylamino)-2H-indolin-2-one in 50 ml of ethanol and from 0.2 g (2 mmol) of imidazole-4-carboxaldehyde, 0.31 g (58%) of 1,3-dihydro-3-(imidazol-4-ylmethylene)-5-(acetylamino)-2H-indolin-2-one is obtained in the form of an orange solid, the characteristic of which is as follows: melting point >260° C.

### Example 5

### Parallel Synthesis of 5-substituted 3-(arylmethylene)-2H-indolin-2-one of General Formula (I)

0.5 mmol of 5-substituted 2H-indolin-2-one of general formula (II), 0.5 mmol of an aromatic aldehyde of general formula (III), 5 ml of ethanol and 1 drop of piperidine are introduced into a heating magnetic reactor with a Zymark condenser of STEM RS2050 type comprising 25 parallel wells each fitted with a 50 ml glass tube. The reaction medium is brought to reflux overnight. After cooling and diluting with 5 ml of water, the precipitate formed is filtered off and dried under reduced pressure. The 5-substituted 3-(arylmethylene)-2H-indolin-2-ones of general formula (I), represented, for example and without implied limitation, by the compounds 5-1 to 5-14, are thus obtained.

### Example 6

### Quantitative Determination of the Inhibition of the p34cdc2/cyclin B Activity by the Compounds According to the Invention

The inhibition of the p34cdc2/cyclin B (CDK1/cyclin B) activity is determined by a protocol which makes it possible to measure the activity for transfer by the enzyme, of a group ³²P, from [γ³²P] ATP to a substrate, histone H1.

CDK-1

+

Test compound

Histone H1+³²P-ADP→³²P-Histone H1+ADP

The preparations of enzymes used correspond either to the starfish p34^{cdc2}/cyclin B enzyme (supplied by L. Meijer, CNRS, Station Biologique [Biological Station], Roscoff, France) or to the human recombinant p34^{cdc2}/Cyclin B enzyme (supplied by New England Biolabs Inc., Beverly, Mass. 01915, USA). The protein Histone H1 (type III-S) is obtained from Sigma.

The buffer C comprises 60 mM of β-glycerophosphate, 30 mM of nitro-phenyl phosphate, 25 mM of MOPS pH 7.0, 5 mM of EGTA, 15 mM of MgCl₂, 1 mM of dithiothreitol and 0.1 mM of orthovanadate. The ATP solution is prepared by mixing 20 μl of (3000 Ci/mmol) [γ³²P] ATP and 90 μl of 1 mM nonradioactive ATP in 890 μl of buffer C.

The reaction medium is prepared according to the following composition:

10 μl of enzymatic preparation are added to 5 μl of Histone H1 (5 mg/ml in buffer C) and to 7 μl of buffer C and mixed. 17.5 μl of the mixture are distributed in the tubes immediately before the test. 3 μl of inhibiting agent to be tested are added to each tube.

The reaction is begun by the addition of 5 μl of the ATP solution, followed by incubating for 15 minutes at 30° C. The reaction is halted by the addition of 0.5 volumes of Laemli 3× buffer. After heating the sample for 5 minutes at 90° C., the samples are subsequently analysed by protein gel electrophoresis, in which the gel comprises 10% of acrylamide, for 1 hour under a voltage of 200 volts using a Novex electrophoresis system. The acrylamide gels are subsequently dried over a Whatman 3MM paper sheet at 80° C. for 1 hour.

The analysis and the quantification of the phosphorylation of the Histone H1 are carried out using an Instant-Imager device (Packard). For each compound concentration tested, the results are expressed as percentage of inhibition of the reaction and are calculated from the untreated enzymatic control.

The concentration of compound that inhibits the phosphorylation reaction of p34^{cdc2}/cyclin B by 50% (IC₅₀) is determined using a semilogarithmic graphical representation of the inhibition values obtained as a function of each of the compound concentrations tested.

Inhibition of CDK1 by the Compounds of the Invention According to the Formula (I)

| | | | CDK1 | |
|---|---|---|---|---|
| | | | % inhibition | CDK1 |
| No. | R5 | Ar | at 10 μM | IC₅₀ in mM |
| 1 | 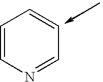 | 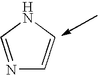 | 99 | 0.3 |
| 2 | 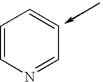 | 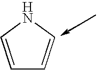 | 87 | 2 |
| 3 | 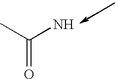 | 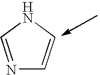 | 100 | 2.5 |
| 4 | 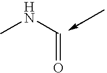 | 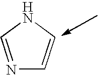 | 100 | 0.7 |
| 5-1 | 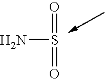 | 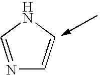 | 86 | |
| 5-2 | 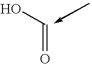 | 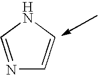 | 85 | |
| 5-3 | Br | 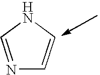 | 100 | 16 |
| 5-4 | 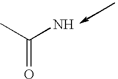 | 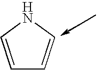 | 80 | |
| 5-5 | 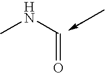 | 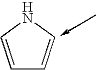 | 99 | |
| 5-6 | 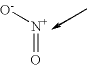 | 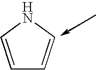 | 91 | 21 |
| 5-7 | 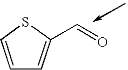 | 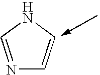 | 100 | 10 |
| 5-8 | 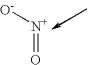 | 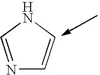 | 81 | 10 |
| 5-9 | 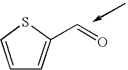 | 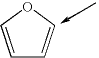 | 75 | |
| 5-10 | 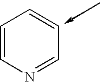 | 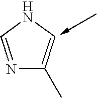 | 100 | 16 |
| 5-11 | 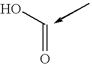 | 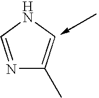 | 100 | 6 |
| 5-12 | 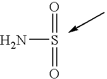 | 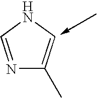 | 100 | 3.5 |
| 5-13 | 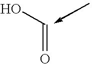 | 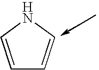 | 79 | |
| 5-14 | 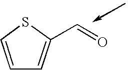 | 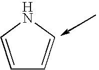 | 62 | |

A compound according to the inventions is regarded as active as an anti-P34^{cdc2}/cyclin B agent when the IC₅₀ is less than 5 μM (5000 nM according to the measurement units used in the table. Several compounds are thus be regarded as inhibitors of the CDK1/cyclin B complex and in particular No. 1.

### Example 7

### Determination of the Inhibition of Clonogenicity

The KB, HCT-116, HT-29, HCT-8, Lovo, PC-3, PC-14, HLF and HLE human cell lines and the C6 rat tumour line originate from the ATCC (American Type Culture Collection, Rockville, USA). The Calc18 human tumour line is a gift from Professor G. Riou (Institut Gustave Roussy, Villejuif, France). The HCT-8, Lovo, PC-3, PC-14, HLF and HLE cells are cultured as a layer in culture flasks in RPMI 1640 medium, L-glutamine 2 mM, penicillin 200 U/mml, streptomycin 200 μg/l, with the addition of 10% of heat-inactivated foetal calf serum. The HCT-116, HT-29, KB, C6 and Calc18 cells are cultured as a layer in a culture flask in Dulbecco's medium comprising L-glutamine 2 mM, penicillin 200 U/ml, streptomycin 200 μg/ml, with the addition of 10% of heat-inactivated foetal calf serum.

The cells in exponential growth phase are trypsinized, washed in PBS 1× and diluted to a final concentration of 5000 cells/ml in complete medium. The test products (in a volume of 50 μl) are added to 2.5 ml of suspension. Subsequently, 0.4 ml of 2.4% Difco Noble Agar, maintained at a temperature of 45° C., is added to the cells. The mixture is then immediately poured into Petri dishes and left at +4° C. for 5 minutes to solidify the agar. The number of cell clones (>60 cells) is measured after incubating for 12 days at 37° C. under a 5% CO₂ atmosphere.

Compound No. 1 is tested at concentrations of 10, 1, 0.1 and 0.01 μM in duplicate. The results are expressed as percentage of inhibition of the clonogenicity with respect to controls. The IC₅₀ is determined graphically from the mean results determined for each concentration of compound.

Inhibition of the Clonogenicity in Agar by Compound No. 1 on Tumour Cell Lines

| Cell line | Tissue of origin | IC₅₀ (μM) |
|---|---|---|
| HCT-116 | colon | 0.28 |
| HT-29 | colon | 1.42 |
| HCT-8 | colon | 1.04 |
| Lovo | colon | 0.9 |
| Calc18 | breast | 1.06 |
| PC-3 | prostate | 0.19 |
| PC-14 | lung | 1.1 |
| HLF | liver | 1.1 |
| HLE | liver | 1.2 |
| C6 | glioblastoma | 10.8 |

A compound is regarded as active as cytotoxic agent if the IC₅₀ is less than 10 μM, which is the case for all the cell lines tested with compound No. 1 (with the exception of C6 rat glioblastoma).

### Example 8

### Measurement of the Inhibition of Proliferation by Compounds No. 1 or No. 2.

The HCT-116 cells are cultured as a layer in a culture flask in Dulbecco's medium comprising L-glutamine 2 mM, penicillin 200 U/ml, streptomycin 2 μg/ml, with the addition of 10% of heat-inactivated foetal calf serum.

The HCT-116 cells in exponential growth phase are trypsinized, washed in PBS 1× and seeded in 96-well microplates (Costar) at the rate of 4×10⁴ cells/ml and 1.5×10⁴ cells/ml (0.2 ml/well), then incubated for 96 hours in the presence of variable concentrations of product to be studied (10, 1, 0.1 and 0.1 μg/ml, each point in quadruplicate). Sixteen hours before the end of incubation, a final concentration of neutral red of 0.02% is added to each well. At the end of incubation, the cells are washed with 1×PBS and lysed with 1% of sodium lauryl sulphate. The incorporation of the dye in the cell, which reflects cell growth, is evaluated by spectrophotometry at a wavelength of 540 nm for each sample using a Dynatech MR5000 reading device.

For each compound concentration tested, the results are expressed as percentage of inhibition of cell growth and are calculated from the untreated control and from the cell-free (blank) culture medium according to the following formula:

(Value compound−value blank/value cell control−value blank)×100.

The concentration of compound that inhibits the growth by 50% (IC₅₀) is determined using a semilogarithmic graphical representation of the inhibition values obtained as a function of each of the compound concentrations tested.

Inhibition of the Proliferation of HCT-116 Cells by Compounds 1 or 2

| | | | HCT-116 |
|---|---|---|---|
| | | | IC₅₀ |
| No. | R5 | Ar | μM |
| 1 | 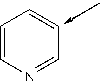 | 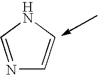 | 0.28 |
| 2 | 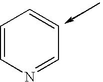 | 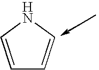 | 8.0 |

A compound is regarded as active as cytotoxic agent if, in one or other of the methods, the IC₅₀ is less than 10 μM, which is the case for both compounds tested in this experiment.

### Example 9

### Determination of the Proapoptotic and Mitotic Blocking Activity Under the Action of Compound No. 1

After exposing HCT-8 cells to compound No. 1 for 48 hours, the cultured cells are trypsinized, washed with 1×PBS and deposited between slide and coverglass in the presence of Hoechst 33342 at a concentration of 1 μg/ml. The percentage of mitotic cells and of cells having apoptotic nuclear bodies is determined by examination and counting of a sample of at least 300 cells distributed over several points of the slide using a fluorescent microscope.

Induction of Apoptosis and Mitotic Blocking Induced by Compound No. 1 on the HCT-8 Line

| Concentration tested | Apoptotic cells | Factor/ | Mitotic cells | Factor/ |
|---|---|---|---|---|
| of compound No. 1 | (%) | control | (%) | control |
| 0 μg/ml (control) | 2.4 | 1 | 6.9 | 1 |
| 0.1 μg/ml | 8.5 | 3.5 | 2.8 | 0.4 |
| 1 μg/ml | 16 | 6.6 | 0.7 | 0.1 |
| 10 μg/ml | 42.5 | 17.7 | 0.4 | 0.06 |

The above example shows a marked correlation between the dose of compound No. 1 tested and

the decrease in cells at the stage of mitosis

the increase in cells in apoptosis.

### Example 10

### Influence of the Action of Compound No. 1 on the Stages of the Cell Cycle

Analysis by flow cytometry makes it possible to demonstrate a blockage in a particular phase of the cell cycle after treatment with a compound.

The HCT-116 cells are seeded in Nunc 6-well dishes. Compound No. 1, at a concentration of 1 μg/ml, is brought into contact with the cells for 4 hours, 1 day, 2 days and 3 days before analysis.

Analysis is carried out using a test with BrDU: (Dolbeare F. et al., Proc. Natl. Acad. Sci. USA, 1983, 80, p. 5573-5577). The cells are treated with 30 μM BrDU for 30 minutes and then trypsinized. After fixing the cells in 1% paraformaldehyde for 16 hours, the latter are digested in pepsin/hydrochloric acid and then rinsed in PBS 1×. Immunolabelling is carried out with an anti-BrDU primary antibody (Becton Dickinson) and a GAM-FITC secondary antibody (Coulter). The DNA is subsequently labelled with propidium iodide in PBS comprising 1 mg/ml of boiled RNAse. This method makes it possible to count the cells in the G1, S and G2M phases.

Study by Flow Cytometry of the Modifications to the Cell Cycle Brought About by Compound No. 1 on the HCT-116 Line

| | | | | Cells in |
|---|---|---|---|---|
| | Contact | Cells in G1 | Cells in S | G2-M phase |
| HCT-116 | time | phase (%) | phase (%) | (%) |
| Untreated cells | 4 h | 70.8 | 16.7 | 10.8 |
| | 24 h | 65.6 | 19.3 | 13.8 |
| | 48 h | 65.0 | 19.9 | 13.5 |
| | 72 h | 65.8 | 20.1 | 12.8 |
| Treated cells (1 μg/ml | 4 h | 62.2 | 20.1 | 16.4 |
| of compound | 24 h | 26.8 | 39.5 | 31.4 |
| No. 1) | 48 h | 7.9 | 30.9 | 58.9 |
| | 72 h | 7.8 | 5.1 | 82.4 |

In the untreated cells, the proportion of cells in G2/M transition is of the order of 10 to 14%, whereas, in the cells brought into contact beforehand with compound No. 1, this level increases until it reaches more than 80% after 72 hours. Thus, in this experiment, virtually all the cells are halted in the G2/M phase by the action of compound No. 1.

1. A compound of formula (I): 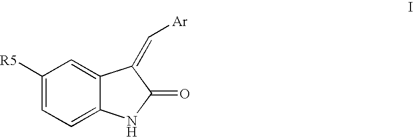
wherein
R5 is selected from the group consisting of 3-pyridyl, 5-pyrimidinyl, —CONH—(C₁-C₄ alkyl), —NHCO—(C₁-C₄ alkyl), halogen, —SO₂NH₂, —NO₂, —CF₃ or thien-2-ylcarbonyl and —CO₂R where R can be hydrogen or C₁-C₄ alkyl; and
Ar is selected from the group consisting of 5-imidazolyl, 2-pyrrolyl optionally substituted by a c1-C₄ alkyl radical, 2-furyl or 2-thiazolyl,
in the e or z geometrical isomeric form or a mixture of the two geometrical isomeric forms.

2. The compound according to claim 1, wherein R5 is a 3-pyridyl or —CONH-methyl or —NHCO-methyl.

3. The compound according to claim 1 or 2, wherein Ar is a 5-imidazolyl or a 5-(4-methylimidazolyl) or a 2-pyrrolyl group.

4. The compound according to claim 1, wherein it is selected from group of formulae consisting of:
1,3-dihydro-3-(imidazol-4-ylmethylene)-5-(pyrid-3-yl)-2H-indolin-2-one;
1,3-dihydro-3-(pyrrol-2-ylmethylene)-5-(pyrid-3-yl)-2H-indolin-2-one;
1,3-dihydro-3-(imidazol-4-ylmethylene)-5-(N-methylcarboxamido)-2H-indolin-2-one; and
1,3-dihydro-3-(imidazol-4-ylmethylene)-5-(acetylamino)-2H-indolin-2-one.

5. A process for preparing the compound according to claim 1, comprising coupling a compound of formula (II): 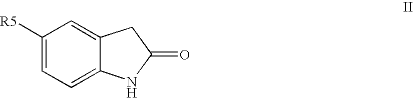
wherein
R5 is selected from the group consisting of 3-pyridyl, 5-pyrimidinyl, —CONH—(C₁-C₄ alkyl), —NHCO—(C₁-C₄ alkyl), halogen, —SO₂NH₂, —NO₂, —CF₃ or thien-2-ylcarbonyl and —CO₂R where R can be hydrogen or C₁-C₄ alkyl,
with a compound of formula (III): 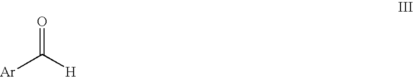
wherein Ar is selected from the group consisting of 5-imidazolyl, 2-pyrrolyl optionally substituted by a C₁-C₄ alkyl radical, 2-furyl or 2-thiazolyl.

6. The process according to claim 5, wherein the reaction is carried out in the presence of piperidine and of ethanol at reflux.

7. A pharmaceutical composition comprising a pharmaceutically effective amount of the compound according to claim 1 in a pharmaceutically acceptable medium.

8. The method of treating cancer in a patient in need thereof, comprising administering to the patient a therapeutically effective amount of the compound according to claim 1.

9. The method of claim 8 wherein the cancer is a primary or secondary tumor.

10. The method of claim 8 wherein the treating arises from the inhibition of a cyclin-dependent kinase.

11. The method of claim 9, wherein the treating arises from the inhibition of CDK-1.

12. The method of claim 8 wherein the treating further comprises combining the treating with radiotherapy, antiangiogenic treatment, or another chemotherapeutic.
